(19) United States
(12) Patent Application Publication
(10) Pub. No.: US 2007/0007512 A1
(43) Pub. Date: Jan. 11, 2007

(54) Bio-inorganic conjugates

(76) Inventors: Nada Dimitrijevic, Downers Grove, IL (US); Tijana Rajh, Naperville, IL (US); Zoran Saponjic, Forest Park, IL (US); Bryan M. Rabatic, Woodridge, IL (US); David M. Tiede, Elmhurst, IL (US); Lin X. Chen, Naperville, IL (US); Peter Zapol, Darien, IL (US)

Correspondence Address: CHERSKOV & FLAYNIK, THE CIVIC OPERA BUILDING, 20 NORTH WACKER DRIVE, SUITE 1447, CHICAGO, IL, 60606, US

(21) Appl. No.: 11/438,180

(22) Filed: May 22, 2006

Related U.S. Application Data
(60) Provisional application No. 60/698,284, filed on Jul. 9, 2005.

(51) Int. Cl. H01L 51/00; C12Q 1/68

(52) U.S. Cl. 257/40; 435/6; 438/1; 977/702

(57) ABSTRACT

A method for producing a bio-inorganic conjugate is provided comprising supplying a plurality of inorganic particles that are axially anisotropic; and positioning biomolecules intermediate the particles to form a chain-like structure. Also provided is an organized microscopic structure capable of vectorial electron transport within the structure, comprising a plurality of inorganic oxide particles, each particle having at least two ends; a first molecule covalently attached to each end to form a plurality of constructs; and a second molecule attached to the first molecule so as to link the constructs and form an elongated substrate.

### BIO-INORGANIC CONJUGATES

This Utility Patent Application claims the benefit of U.S. Provisional Patent Application No. 60/698,284 filed on Jul. 9, 2005.

### CONTRACTUAL ORIGIN OF THE INVENTION

The United States Government has rights in this invention pursuant to Contract No. W-31-109-ENG-38 between the University of Chicago and Argonne National Laboratory.

### BACKGROUND OF THE INVENTION

1. Field of the Invention

The present invention relates to nanoscopic charge carriers and the method for manufacturing microscopic charge carriers, and more particularly, this invention relates to the combination of inorganic and organic moieties to arrive at electron transport structures and a method for manufacturing such structures.

2. Background of the Invention

Exceptional electronic, optical, chemical and biological activities stem from materials scaled to nanoscaled dimensions, for example, dimensions ranging in size to less than 1000 nanometers.

The inventors have previously reported on the synthesis of semi-conductor particles having varied physical morphologies and interesting surface properties and reactivity. These reports are found in N. M. Dimitrijevic, et al., J. Am. Chem. Soc. 2005, 127, pp 1344; Z. V. Saponjic et al., Adv. Mater., 2005, 17, pp 111; T. Rajh, et al., J. Phys. Chem. B, 2002, 106, 10 543; and T. Paunesku et al., Nat. Mater., 2003, 2, 343, all incorporated herein by reference.

U.S. Pat. No. 6,667,606 B1 awarded to some of the inventors, and incorporated herein by reference, discloses nanoparticle:biomolecule composites exhibiting charge transfer characteristics at various excitation levels.

A need exists in the art for nanoscaled structures of definite configuration which facilitate vectorial charge movement. The structures should be reproducible in fabrication, and physically manipulatible in situ, and even in vivo. The structures, as subunits also should allow for scaffolding to thereby provide larger, homogeneously built constructs to confer vectorial transport of electrical charge over large distances.

### SUMMARY OF THE INVENTION

An object of the present invention is to provide an inorganic-organic construct to facilitate electron transfer, and a method for producing such a construct, which overcomes many of the shortcomings of the prior art.

Another object of the present invention is to provide a metal oxide surface between 300 nm and 400 nm long and between 40 nm and 60 nm wide for coupling with organic moieties. A feature of the surface is that it is produced without contaminants or other matter on its surface. An advantage of the invented surface is that it facilitates direct contact with the organic moieties, thereby enhancing electrical communication therebetween.

Yet another object of the present invention is to provide an electrical switch comprising inorganic metal oxide and organic compounds. A feature of the invention is that the switch is activated when exposed to radiation of a predetermined frequency. An advantage of the invention is that the switch is small enough to be used in situ and in vivo to direct electron flow to targeted tissues.

Briefly, the invention provides a method for producing a bio-inorganic conjugate comprising supplying a plurality of inorganic particles that are axially anisotropic; and positioning biomolecules intermediate or in between the particles to form a predetermined shape. The shape can be, but is not limited to, a chain of repetitive linking subunits, such as an aggregate of rods forming a chain. Alternatively, a chain or aggregate of different shaped subunits is suitable. As discussed below, rods, branched chain structures, polygonals such as stars, cubes, triangles, and a combination of these shapes are suitable.

Also provided is an organized microscopic structure capable of vectorial electron transport within the structure, comprising a plurality of inorganic oxide particles, each particle having at least two ends; a first organic complex (such as a dopamine-biotin complex) covalently attached to each end of the particle to form a plurality of constructs; and a second molecule (such as avidin) attached to the first organic complex so as to link the constructs and form an elongated substrate.

The invention further provides a method for fabricating semiconductor particles, the method comprising supplying a semiconductor feedstock substrate shaped as a tube; and subjecting the substrate to predetermined temperatures pressures and pH for a time sufficient to produce single crystal particles emanating from surfaces of the tube, wherein each of the crystal particles define at least a first termination point and a second termination point, wherein the termination points define irregular or alternate crystal lattice structure.

Also provided is an electrical switch comprising a first inorganic semiconductor particle having a first end and a second end; a complex of organic molecules attached to the first end and the second end to form an inorganic-organic construct having a first terminus and a second terminus; whereby the semiconductor induces a positive charge on the complex when the semiconductor is subjected to illumination, other radiation or some other means to promote ionic excitation within the semiconductor; and a second semiconductor particle attached to the first terminus, wherein the second semiconductor particle detaches from the first terminus when the semiconductor is subjected to radiation.

### DESCRIPTION OF THE DRAWING

The present invention together with the above and other objects and advantages may best be understood from the following detailed description of the embodiment of the invention illustrated in the drawing, wherein:

FIG. 1 is a schematic diagram of the invented bio-inorganic conjugate and energy flow, in accordance with features of the present invention;

FIG. 2 is a reaction sequence depicting the joining of various moieties of the invented construct, in accordance with features of the present invention;

FIG. 3 are transmission electron micrographs (TEM) of various structures formed with the joining of the invented conjugate construct, in accordance with features of the present invention;

FIG. 4 is EPR spectra comparing charge separations on titanium-dopamine-biotin complexes and titanium-dopamine-biotin-avidin complexes, in accordance with features of the present invention;

FIG. 5 is a schematic depiction of electron and hole transport in the invented construct during radiation exposure, in accordance with features of the present invention;

FIGS. 6 A-C are schematic depictions of cleavage of the invented construct when exposed to radiation of a predetermined wavelength, in accordance with features of the present invention; and

FIGS. 7A-B are graphs showing changes in electrical potential of the invented inorganic-organic electrode, in accordance with features of the present invention.

### DETAILED DESCRIPTION OF THE INVENTION

The present invention provides an inorganic-organic module to enable the vectorial transport of electrons along the module and from one constituent of the module to another in a predetermined direction. The constituents are either covalently or noncovalently linked to each other. Also provided is a method for producing the construct, and elongated structures comprising repeating sequences of the construct. While sizes of the elongated structures will vary, initial experimental data shows that the structures can be at least 300 nm long and 50 nm wide.

A salient feature of the module is the use of a semi-conductor particle which has various electronic ground and excited states. The particle is combined with biological molecules or other organic material to produce the module. Modules are positioned end to end to form an elongated construct. Upon excitation, and through a series of covalent interactions between the particle and a first molecule, and noncovalent interactions between the first molecule and a second molecule, the construct is capable of transport of electrons from the particle of a first module to a region of the aforementioned elongated structures which is remote from the first module.

Specifically, the instant invention teaches the linking of elongated rods of TiO₂, which have preferential chemical activity at the their tips. This chemical activity is the result of defects or alterations in the crystal lattice of the metal, as more fully discussed by the inventors in B. M. Rabatic, et al., Advanced Materials, 18, pp 1033-1037 (2006) and incorporated herein by reference. Linking is effected via noncovalent protein:biomolecule interaction (e.g., avidin:biotin conjugation) to create a chain like structure or construct. Generally, the interaction is the product of Van derWaals forces, hydrogen bonding, and/or a combination of these forces. Photo-induced charge transfer in these hybrid chains result in a change in redox state of the proteins. This redox change results in a modification of the binding abilities of avidin to biotin.

As such, the construct is capable of inducing site-specific redox chemistry in TiO₂— bound proteins, resulting in biological catalysis. This catalysis is the result of light-induced charge separation within the construct.

Just as surprising and unexpected, the inventors have noted that light of predetermined wavelength cleaves the links between the elongated rods. This allows the cleaved end of the rod to rotate freely to either reestablish electrical contact with the remainder of the construct at a later time, or else establish a new electrical contact with an adjacent construct. This photo-induced cleavage and reattachment of linkages between subunits of the construct is a means for providing an electrical switch along a construct or between constructs which are in close spatial relationship to each other.

An embodiment of the invention involves immobilization of avidin onto thin film (approximately 200 to 400 nanometers nanocrystalline TiO₂ on indium-tin oxide (ITO) was obtained via avidin-biotin binding. In the construction of these organic-inorganic constructs, dopamine was used as the TiO₂ surface-active ligand providing a conductive lead to covalently linked biotin. Each layer of a biomolecule subunit (dopamine, biotin, avidin) attached to the TiO2 film produced an increase in overpotential (η) on the remaining electrode. An increase in overpotential of Δη 400 mV was measured for a monolayer of avidin bonded to biotinylated electrodes.

The absorption of light by inventor-fabricated nanocrystallites results in charge separation, with holes (positive charge) being localized on the avidin. This photo induced charge separation and oxidation of avidin results in the dissociation of the avidin-biotin complex, promoting changes in the photoelectroactivity of avidin-modified molecules.

Particle Detail

Inorganic nanoparticles configured as rods, and ranging in length of between 200 nanometers (nm) and 500 nm and ranging in diameter of 40 nm and 80 nm were provided. A myriad of metal oxides are suitable nanoparticles candidates, including, but not limited to TiO₂, WO₃, Fe₂O₃, ZrO₂, SnO₂, VO₂ and combinations thereof.

Surprisingly and unexpectedly, the inventors found site-specific defects located at the tips of the synthesized rods. Hereinafter referred to as “corner defects”, these anomalies are related to the size and shape of features on the particle.

The site-specific defects include a deviation from the hexa-coordinated (Octahedral) configuration of the metal atoms in the lattice such that a constraining of the atomic arrangement of the atoms occurs. This confinement occurs within less than 10 atomic layers from the tip of the synthesized particle, resulting in an under-coordinated (i.e., less than the normal Oxygen-atom contingent) atomic character to the TI metal sites. This under-coordinate causes a lengthening of the Ti-Ti distances along the longitudinal axis of the crystal.

The incompletely coordinated Ti defect sites exhibit a high affinity for oxygen-containing ligands and present the opportunity for chemical modification. For example, and as more fully disclosed in Saponjic et al., Adv. Mater. 2005, No. 8, pp 965-971 and incorporated herein by reference, oxygen-rich enediol ligands form strongly coupled conjugated structures by repairing the coordination surface via chelation. As a consequence, the intrinsic properties of the semiconductor change and new, hybrid molecular orbitals are generated by mixing the orbitals of chelating ligands and the continuum states of the metal oxides. This results in the red-shift of the absorption compared to unmodified nanocrystallites.

These conjugated structures can be manipulated and connected tip-to-tip to form “chainlike” structures. The formation of such chainlike structures is found in Dimitrijevic et al., J. Am. Chem. Soc. 2005, 127 pp 1344-1345, heretofore incorporated herein by reference.

Fast Fourier transformation (FFT) analysis quantified the lattice spacing anomalies. Specifically, the tip defects manifested as an increase of approximately 0.3 Angstroms (Å) to the Ti—Ti-bond length of the defect area when compared to the bulk material. At the defect, the Ti—Ti spacing is 3.96±0.20 Å, whereas the non-defective spacing measures 3.70±0.19 Å, as disclosed in Rabatic et al., Adv. Mater., 2006, 18, pp 1033-1037, heretofore incorporated herein by reference.

Rod Formation Detail

The inventors formulated and utilized a surfactant-free hydrothermal procedure to form the anatase TiO₂ rods. Specifically, the inventors developed a one-pot synthesis method of high quality titanium-dioxide nanocrystals of different shapes and sizes using titania nanotubes as precursors in a hydrothermal process, which is to say a process involving a predetermined application of temperature and aqueous solution (the later manifesting target pH values).

The inventors found that titania nanotubes are ideal starting materials for reshaping because of the adequate ratio of surface and bulk (interior and exterior) under-coordinated sites that present changes in the coordination of surface Ti atoms from octahedral (D_{2d}) to square pyramidal structures (C_{4V}). The nanotubes were fabricated pursuant to the method disclosed in the Adv. Mater. 2005 paper heretofore incorporated by reference.

The inventors synthesized axially anisotropic nano-objects such as nanorods and starlike nanoparticles, axially isotropic faceted nanoparticles, bricks and prismatic nanoparticles, all without surface modifiers. The absence of surface modifiers is noteworthy for providing direct contact of organic moieties to the surface of the metal oxide.

Perfect anatase crystal structures were produced, i.e., crystals without any substantial amounts of structural disorder. Rather, virtually all structural anomalies are observed at the tips of the rods, stars, corner of cubes and surfaces of spheres. These transformations of shape and size during synthesis are caused by changes in temperature, pressure and by changes in the surface environment (charge) by changing the starting pH of the water solution. As such, the instant invention provides a method for fabricating semiconductor particles (neat) having predetermined shapes.

Ellipsoidal Shape

Rod Fabrication Detail

Titania nanorods of ellipsoidal shape were synthesized by a hydrothermal method using 0.03 M water dispersion of titania nanotubes, pH=7 as starting materials. During 2h in autoclaving conditions at 250° C., TiO₂ nanotubes (5.8±0.1 Å layer thickness, an outer diameter of about 10-12 nm, and few hundred nanometers in length) were transformed into TiO₂ rods with increased crystalline domain and dimensions (whereby the 5.8 Å layer is the domain of crystallinity). TEM images of partially grown nanorods show that the growth of TiO₂ nanorods occurs perpendicular to the exposed surface of nanotubes surfaces by recrystallizing nanotube material into a fully developed anatase crystalline lattice.

The growth process is over when substantially all available nanotube material is converted into nanorods. The diameter of fully grown nanorods is 70 nm and the length is 300-600 nm. Nanoparticles of different aspect ratios (e.g., d=30 nm, whereby widths are from 50-80 nm and lengths are up to 550 nm) are synthesized by increasing the starting concentration of titania nanotubes and decreasing reaction time. This shape-change from tubes to rods is followed by the surface structure change. The majority of under-coordinated surface defect sites that are located along the walls of the nanotubes disappear, only those located at the surface tip of the nanorods remain.

Due to enhanced chemical reactivity of spatially isolated defect sites on the tip of the nanorods, preferential binding and control of site-specific redox chemistry of nanorods is obtained. As a consequence, titania nanorods can be oriented into organized structures, for example wires, switches, capacitors, etc., which are capable of electron capture/storage and vectorial electron transport.

Star-Shape Particle

Fabrication Detail

Synthesis of multi-apex (i.e. starlike) TiO₂ nanoparticles required additional treatment of titania nanotubes before applying the hydrothermal method. Water dispersion of TiO₂ nanotubes, pH=7, was refluxed to remove OH⁻ ions adsorbed on the surface and intercalated into tube-like structures until pH reached pH=11-12. After centrifugation the supernatant was discarded and a new amount of water, pH=7, was added. This procedure was repeated three times. After the last exchange of water, dispersion was ready for the hydrothermal process of 2h at 250° C. These places on the surface are centers of nucleation for the growth of starlike particles (d=300 nm).

The length of the star tentacles can be controlled by changing the reaction time and the starting concentration of nanotubes. An increase in concentration together with increased reaction time increases the number of nucleation centers, and thus the number of stars and the length of star tentacles or apexes. These types of TiO₂ nanoparticles could be used as building blocks for synthesis of highly porous photo catalytically active film for redox processes in the gas phase.

Miscellaneous Geometric

Shape Fabrication Detail

Brick-like TiO₂ nanoparticles (80×120 nm) were synthesized via hydrothermal treatment of concentrated suspension of nanotubes at pH7 under the same condition used for the growth of nanorods. For that purpose, a three times larger aliquot of nanotubes was dialyzed to pH=7, added into the water and hydrothermally treated for 2h at 250° C.

Faceted TiO₂ nanoparticles (d=25-30 nm) were synthesized also by applying hydrothermal methods on suspension of titania nanotubes in the proton rich aqueous system. “Faceted” is taken here to describe nano scaled particles having crystalline facets or regions exposed to the reaction solution. In this synthetic procedure, pH of dispersion of the same concentration as used in the synthesis of rods and starlike particles, was decreased until pH=2, followed by a 2 h hydrothermal process at 250° C. After synthesis the powder is efficiently redispersed in water giving a transparent colloidal solution of TiO₂ nanocrystals suitable for any type of optical measurement.

A prismatic shape of TiO₂ nanoparticles (150-650 nm) was obtained by dissolving nanotubes or nanorods in concentrated sulfuric acid for 12 h, centrifuged and washed with pure water four times and resuspended in water.

The general strategy described for synthesis of TiO₂ nanocrystals of different shapes and sizes using nanotubes as precursors can be applied for doping titania with a variety of transition metals with the aim to alter their optical and magnetic properties and enhance their photo catalytic activity.

The inventors identified the molecular structure and reactivity of local surface sites associated with corner, edge and high curvature interfaces. For this purpose EPR spectroscopy was used to study low temperature electron transfers in differently shaped TiO₂ nanoparticles. It was found that a different distribution of electron density in the TiO₂ nano objects exists after illumination. Charge separation in nanoparticles that have diameters smaller than the exciton radius do not show the existerice of lattice electrons, suggesting that charges never separate after strong (excitonic) interaction, and the majority of charges that are formed disappear in recombination. Very similar behavior was found for nanotubes that consist of 5.8 Å layers of anatase TiO₂ rolled nanotube structures. After photo excitation, only a small fraction of electrons was able to escape excitonic interaction and localize at the surface trapping sites. As the size of the nano objects exceeds the exciton diameter (30 Å), excitonic interaction is followed by separation of charges and the characteristic EPR spectrum of lattice trapped electrons is observed.

The inventors found that the local environment of localized electrons strongly depends on the shape of the nano objects. The nanorods show strong localization of electrons at high curvature sites—tips—and display the same Ti(III) environment as high curvature spherical nanoparticles in contrast to faceted nanoparticles that have signals similar to single crystal anatase. This leads to the conclusion that tips of the nanorods have the lowest excitation energies when functionalized with enediol ligands.

In one embodiment, the particles generated are single crystals and have a rod-like configuration with lengths up to 500 nm and widths up to 80 nm. The production and utilization of single crystals is noteworthy inasmuch as no grain boundaries exist, which would otherwise affect/stymie charge transport. As such, the generated particles lack grain boundaries and other internal structures, thereby providing unimpeded charge conduits within the bulk of the crystal.

Also surprisingly and unexpectedly, the inventors found that the under-coordinated defect sites facilitate direct chemical functionalization and specifically, the Ti—Ti atom positioning in the defect site represents an optimal docking site for the enediol groups of dopamine. As such, the surface tip defect promotes the binding of dopamine exclusively to the tips of the synthesized titanium particle.

The Ti-dopamine construct serves as a building block for an elongated substrate, each building block attached via a biotin-avidin complex. Specifically, a biotin molecule having a first end bound to dopamine, has a second end bound noncovalently to two docking sites at a first end of an Avidin molecule. (Avidin has four identical binding sites for biotin thereby facilitating linkage of up to four Ti-dopamine constructs.)

Ti-Dopamine-Biotin-Avidin

Construct Detail

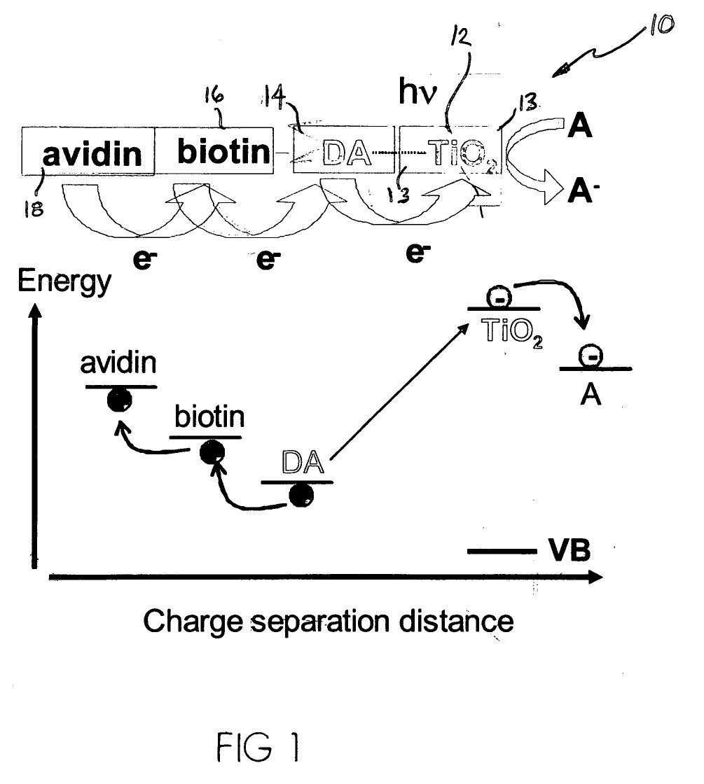

FIG. 1 schematically depicts the final Ti-dopamine-biotin-avidin construct, designated generally as 10, and the electron vectoring phenomenon resulting therefrom. Length of two biotin derivatives used in construct ranged from 13 to 25 angstroms (Å). It should be noted that while biotin and avidin are the biomolecules utilized in this illustration, the invention is also applicable to other similar type biologicals. As such, specific moiety lengths and diameters of semi-conductor particles are provided herein for illustrative purposes only and to limit the scope of the invented system. Generally, suitable biomolecules include, but are not limited to avidin, streptavidin, biotin, various biotin analogues such as iminobiotin, desthiobiotin, LC-biotin, and combinations thereof.

First, the TiO₂ particle 12 is provided, having the corner defects discussed supra. The corner defects facilitate covalent bonding with dopamine 14 via a bidentate complex of dopamine OH groups with the under-coordinated TI surface atoms. Upon bonding with dopamine (one titanium atom to two hydroxyl groups on the dopamine), the constrained configuration of the Titanium atoms involved relax to the original octahedral lattice configuration, resulting in the formation of a very stable ligand-to-metal complex, estimated at 25 kcal/mole. This relaxation serves as a means for eliminating surface trapping centers which would otherwise constrain mobile electrons.

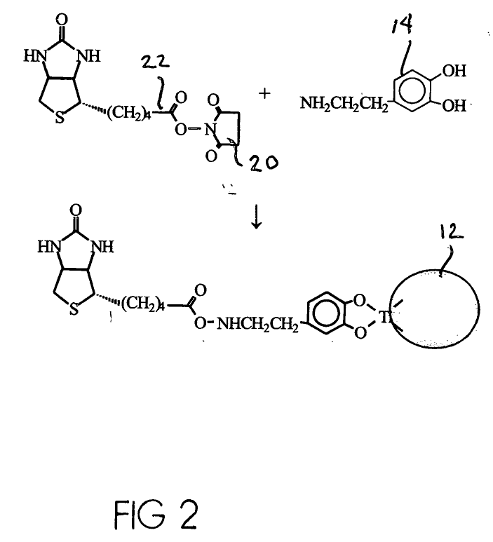

This dopamine preparation of the tips 13 of the Titanium particle facilitates covalent bonding of biotin 16 to titanium particle via an intermediately positioned dopamine moiety, via a condensation reaction, as depicted in FIG. 2. Alternatively, dopamine can first be bound to biotin to form a dopamine-biotin construct, with that construct then bound to the constrained sites of titanium.

In a first step, the succinimidyl group 20 on the end of the valeric chain 22 of biotin is replaced with dopamine through the later's terminal amino group.

The assembling of TiO₂ protein hybrid architectures was performed using a procedure disclosed in Dimitrijevic, N. M. et al., J. Am. Chem. Soc., Vol 127, pp 1344 (2005) and incorporated herein by reference. One part of biotinylated TiO₂ nano-rod solution, phosphate buffer pH=7, was mixed with excess avidin and incubated overnight. The unbound avidin was washed out by repeated centrifugation, decanting, and washing with water. The resulting solution of the concentrated TiO₂-dopamine-biotin-avidin was mixed with additional TiO₂-dopamine-biotin moiety and incubated for a few hours. The resulting binding of avidin with biotin produces almost exclusively tip-to-tip assembly of TiO₂ rods.

The formation of the TiO₂-dopamine-biotin-avidin construct arises from the high affinity of avidin-biotin binding that involves multiple hydrogen bonds, van der Waals interactions between biotin and avidin, and the ordering of surface polypeptide loops that bury the biotin in the protein interior.

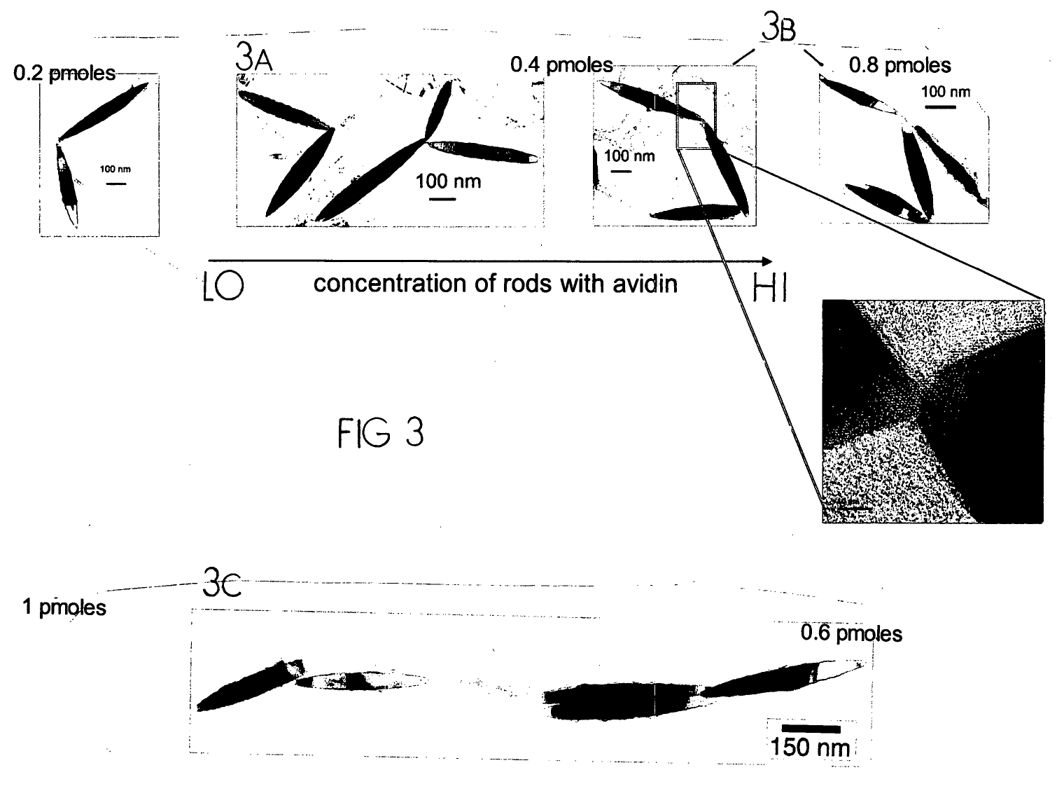

As depicted in FIGS. 3A-D, the number of attached rods depends on the ratio of concentrations. When low ratios are employed, scaffolding of the constructs occurs whereby doublets (FIG. 3A) and triplets (FIG. 3B) form. Increasing the concentration of added TiO₂/DA-biotin-avid hybrids results in the formation of more complex structures such as elongated substrates, as depicted in FIG. 3C. Generally, an increase in the concentration of avidin increases production of elongated rod-like structures.

Charge Transfer Detail

Previously, the inventors identified Ti³⁺ and dopamine+ as radical species formed upon photo-excitation of the TiO₂/dopamine complex. See, for example, U.S. Pat. No. 6,677,606 issued to the Assignees on Jan. 14, 2004, and incorporated herein by reference.

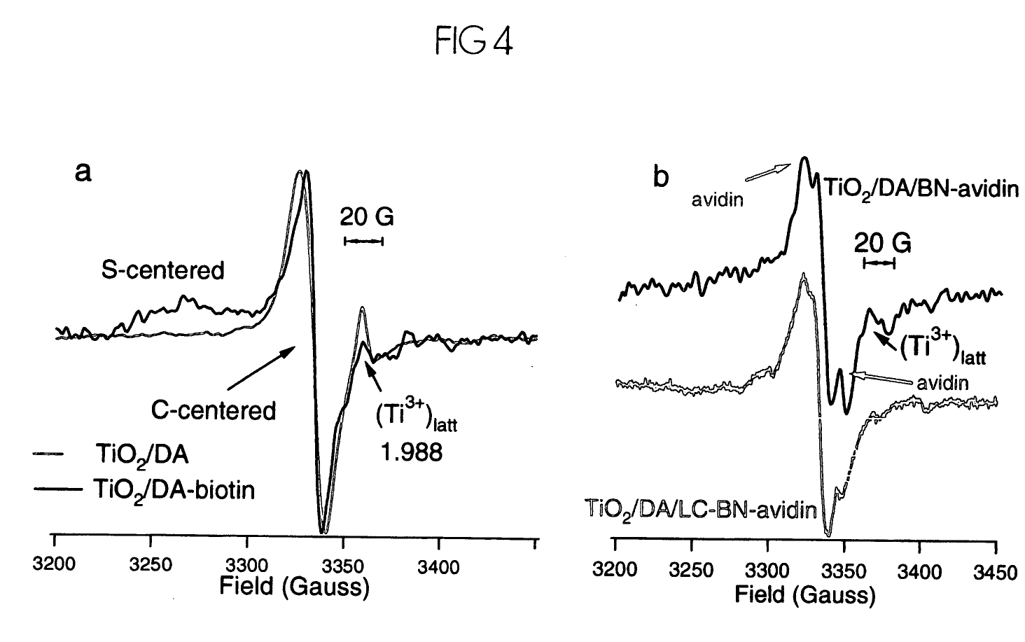

Normalized X-band EPR spectra for the charge separations experienced by the invented constructs are depicted in FIGS. 4A and 4B The spectra were obtained at 4.6 K after illumination (Xe 300 W lamp) of TiO₂/DA (red line) and Ti₂/DA-biotin (black line), as depicted in FIG. 4A. The results of illumination of TiO₂/DA-biotin-avidin hybrids are depicted in FIG. 4B, wherein the black line corresponds to biotin and the blue line corresponds to long chain biotin (LC-biotin) which is Biotinyl-6-aminocaproic acid. Measurements were conducted at 9.0 GHz.

Surprisingly and unexpectedly, with biotin conjugated to the pendant side chain of dopamine, the photogenerated electrons and holes from the original TiO₂-dopamine construct separate further, such that holes localize at the biotin moiety and the electron is on the titanium particle. This is depicted in FIG. 4A. Charge is likely localized on the thiophene ring of the biotin, with oxidation occurring at the C-2 position. Generally, stability on the construct is conferred with avidin donating electrons to counteract or neutralize the photoexcited TiO₂.

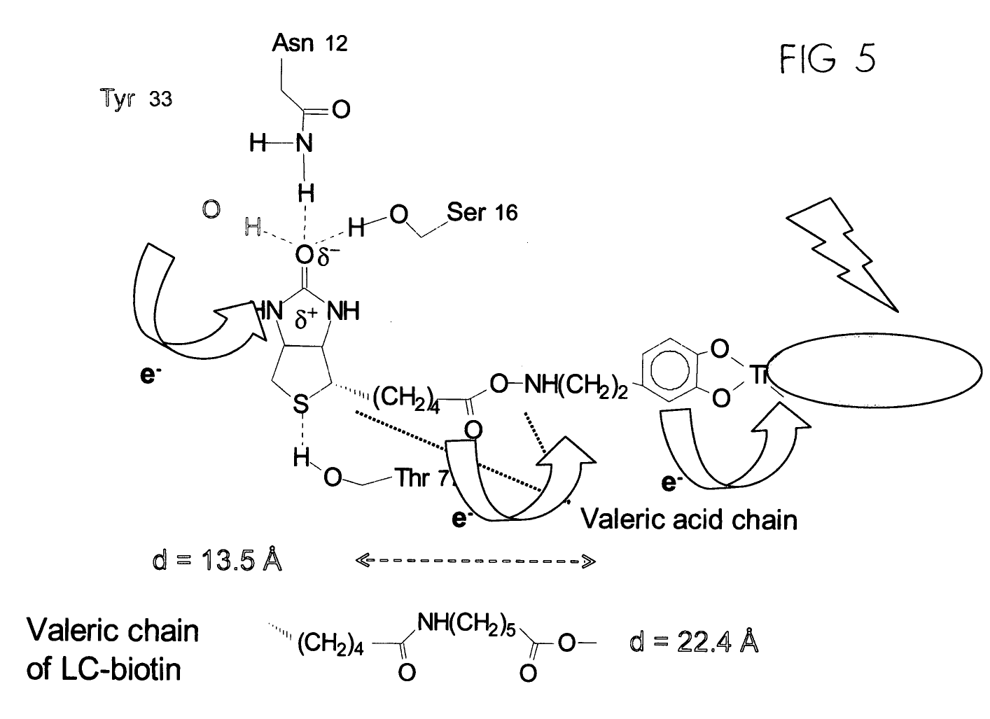

When TiO₂-dopamine-biotin-avidin hybrids are photoexcited, transfer of photogenerated holes occurs from TiO₂ to avidin (see FIG. 4B). Oxidation of the avidin probably occurs at the Tyr33 so as to form a critical hydrogen bond with the biotin. Tyrosine and tryptophane are two amino acids in avidin prone to oxidation, with tyrosine having a more negative redox potential and thus easier to oxidize. A schematic diagram of the electron transport mechanism is depicted in FIG. 5.

Specifics of the photo excitation process are found in U.S. Pat. No. 6,677,606 B1, and incorporated herein by reference.

A myriad of excitation means are utilized including wavelength from xenon light, UV light, typical visible light within the spectrum defined from 0.7 microns (μm) to 0.4 μm, and combinations thereof. Generally, radiation contacts the titanium-dopamine complex for a time sufficient to cause electron movement from the metal's valence band to its conduction band. This generates holes on as far back as the avidin moiety which is noncovalently attached to the semiconductor.

Chain Separation Detail

The inventors have discovered that certain photo-excitation energies cleave the avidin biotin bond in the construct. Specifically, the absorption of light by the semiconductor metal results in charge separation, with holes being localized on avidin. The photo induced charge separation and oxidation of avidin yields to the dissociation of the avidin-biotin complex, promoting changes in the photoelectroactivity of the avidin-modified electrodes. References to “electrodes” in this specification include the substantially entire construct, which is to say the semi-conductor particle in electrical communication with the dopamine in electrical communication with the biotin in electrical communication with the avidin. As such, the electrode is a construct whereby the semi-conoductor particle is in electrical communication with avidin.

When this cleavage occurs along a tip-to-tip assembly of the organic-inorganic conjugate subunits such as those depicted in FIG. 3C, two free ends, intermediate the terminal ends of the assembly, are produced. This allows the effected organic-inorganic subunits to rotate, pivot, or otherwise move freely about their still attached bond located proximal to the bond breakage.

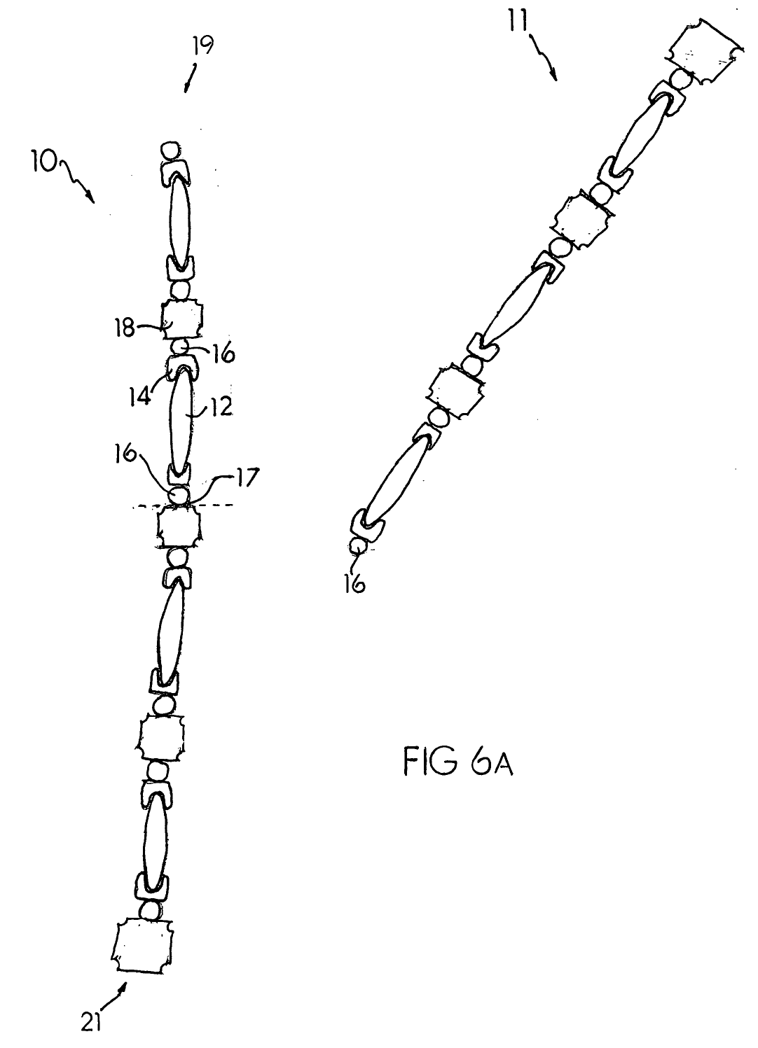

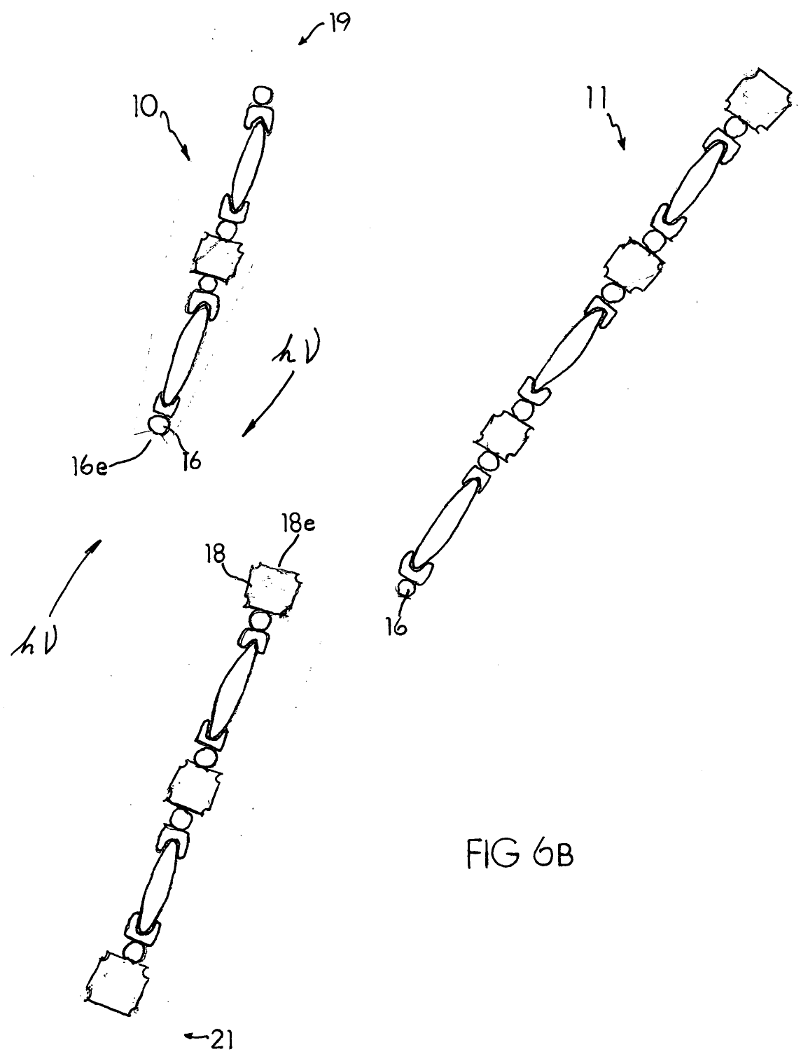

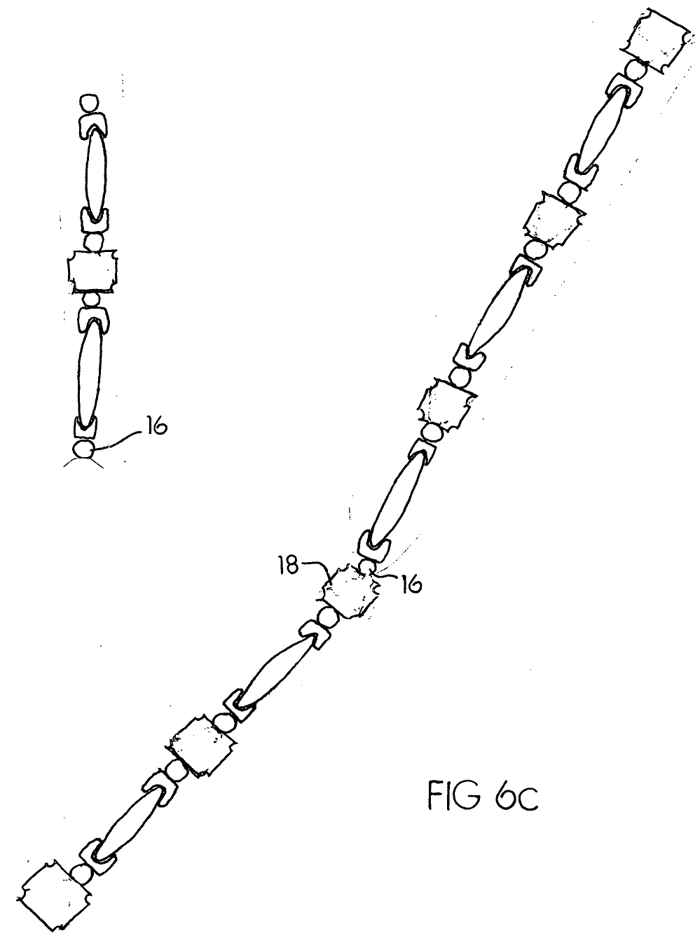

A schematic depiction of the aforementioned nanoswitch is found in FIG. 6. FIG. 6A depicts two separate constructs 10, 11 positioned in close spatial relationship to each other. Each of the constructs are depicted having a first terminating end 19 and a second terminating end 21. Intermediate the first and second terminating ends are subunits continually arranged as depicted in FIG. 1.

When light hV of a suitable wavelength (e.g., 300 nm to 700 nm) interacts so as to excite the first construct 10, cleavage of the construct occurs at a selected biotin-avidin juncture 17. This cleavage is depicted in FIG. 6B. As a result of this cleavage, two additional free ends are produced, namely a first intermediate biotin terminus 16e (which terminates with a biotin moiety) and a second intermediate avidin terminus 18e which terminates with an avidin moiety.

Upon relaxation of the electronic state of the construct 10 (i.e., when illumination or other cleavage-inducing radiation is withdrawn), either reestablishment of the biotin-avidin juncture 17 occurs or else a new link-up with a heretofore unassociated complex 11 occurs. Link-up with an associated complex is depicted in FIG. 6C wherein the intermediate avidin terminus 18e is shown in linkage with a biotin moiety 16 of the adjacent construct 11.

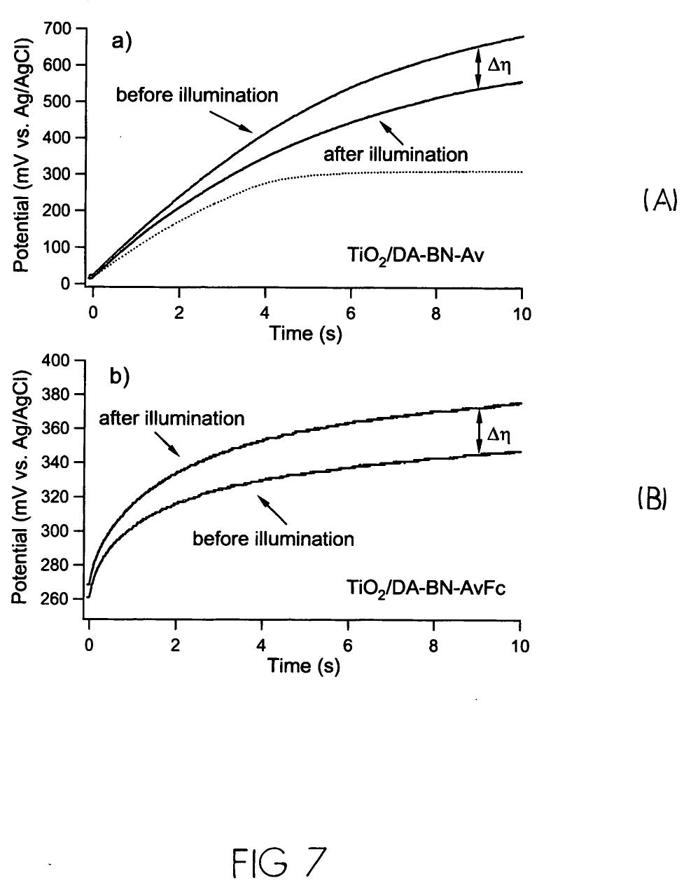

Dissociation of the avidin-biotin noncovalent complex is shown in FIGS. 7A and 7B. The inventors found that cleavage of the noncovalent avidin-biotin linkage is the result of a charging of avidin via oxidation of its tyrosine and tryptophan given that both are amino acids of avidin having the most preferable redox potential for oxidation. Tyr 33 is especially implicated given its involvement with avidin's binding to biotin. In an environment devoid of a redox couple to scavenge the photogenerated charges, chemical reactions are induced within the hybrid, causing disengagement of avidin from biotin. Under an open circuit condition, this disengagement of avidin is evidenced by a lowering of surface charge on the remaining electrode, in other words, a lowering of potential (resistance) at the surface, FIG. 7A.

Conversely, FIG. 7B shows a lowering of resistance in the presence of a redox couple attached to avidin (compared to native avidin), in this case ferrocene-labeled avidin (AvFc). When the redox moiety is removed from the electrode surface to the bulk of the solution environment, as a result of photo induced charge transfer, the decrease in efficiency of interfacial electron-transfer reactions increases the overall resistance. Partial removal of avidin from electrodes upon illumination results also in the decrease in photo current signals.

Illuminating the modified electrodes (the electrodes heretofore described as substantially the entire inorganic-organic charge transport construct) with light (e.g. white light) generates charge residues on the complex. Prior to illumination, the electrodes are immersed in an oxygen rich environment, such as oxygenated phosphate buffer solution, so as to facilitate the scavenging of photogenerated electrons, thereby allowing accumulation of holes on the visiting protein (e.g. avidin).

While the invention has been described with reference to details of the illustrated embodiment, these details are not intended to limit the scope of the invention as defined in the appended claims.

1. A method for producing a bio-inorganic conjugate comprising:
c) supplying a plurality of inorganic particles that are axially anisotropic; and
b) positioning biomolecules intermediate the particles to form a predetermined-shape structure.

2. The method as recited in claim 1 wherein each of the inorganic particles are elongate so as to define a longitudinal axis and at least two ends.

3. The method as recited in claim 2 wherein each of the ends displays chemical activity specific for the biomolecules.

4. The method as recited in claim 1 wherein the biomolecules are compounds selected from the group consisting of avidin, streptavidin, biotin, various biotin analogues such as iminobiotin, desthiobiotin, LC-biotin, and combinations thereof.

5. The method as recited in claim 1 wherein steps a and b are repeated until the structure is approximately 1000 nanometers (nm) in length.

6. The method as recited in claim 1 wherein the inorganic particles are oxides selected from the group consisting of TiO₂, WO₃, Fe₂O₃, ZrO₂, SnO₂, VO₂, and combinations thereof.

7. A bio-inorganic conjugate produced by the method recited in claim 1.

8. An organized microscopic structure capable of vectorial electron transport within the structure, comprising:
a) a plurality of inorganic oxide particles, each particle having at least two ends;
b) a first molecule covalently attached to each end to form a plurality of constructs; and
c) a second molecule attached to the first molecule so as to link the constructs and form an elongated substrate.

9. The structure as recited in claim 8 wherein the ends are modified to facilitate attachment of the first molecules.

10. The structure as recited in claim 8 having a length of between 500 nanometers and more than one micron.

11. The structure as recited in claim 8 wherein a bidentate molecule is positioned intermediate the oxide particle and the first molecule.

12. The structure as recited in claim 11 wherein the bidentate molecule is dopamine and wherein spacing between enediol groups of the dopamine match spacing of titanium atoms located at the ends.

13. The structure as recited in claim 8 wherein the first molecule is biotin and the second molecule is avidin.

14. A method for fabricating semiconductor particles, the method comprising:
a) supplying a semiconductor feedstock substrate shaped as a tube; and
b) subjecting the substrate to predetermined temperatures, pressures and pH for a time sufficient to produce single crystal particles emanating from surfaces of the tube, the particles defining at least a first termination point and a second termination point.

15. The method as recited in claim 14 wherein the termination points define irregular crystal lattice structure.

16. The method as recited in claim 14 wherein the substrate is comprised of TiO₂.

17. The method as recited in claim 14 wherein the particles define a geometric shape selected from the group consisting of rods, prisms, ellipses, spheres, stars, cubes, and pyramids.

18. An electrical switch comprising:
a) a first inorganic semiconductor particle having a first end and a second end;
b) a complex of organic molecules attached to the first end and the second end to form an inorganic-organic construct having a first terminus and a second terminus; whereby the semiconductor induces a positive charge on the complex when the semiconductor is subjected to radiation; and
c) a second semiconductor particle attached to the first terminus, wherein the second semiconductor particle detaches from the first terminus when the semiconductor is subjected to radiation.

19. The electrical switch as recited in claim 18 further comprising a third semiconductor particle attached to the second terminus.

20. The electrical switch as recited in claim 18 whereby the semiconductor is a single crystal.
